SEQUENCE LISTING (5 sequences)

SEQ ID NO: 1 - protein, 76 aa, Homo sapiens [sequence omitted]

SEQ ID NO: 2 - protein, 605 aa, Homo sapiens
MSQLSSTLKRYTESARYTDAHYAKSGYGAYTPSSYGANLAASLLEKEKLGFKPVPTSSFLTRPRTYGPSSLLDYDRGRPLLRPDITGGGKRAESQTRGTERPLGSGLSGGSGFPYGVTNNCLSYLPINAYDQGVTLTQKLDSQSDLARDFSSLRTSDSYRIDPRNLGRSPMLARTRKELCTLQGLYQTASCPEYLVDYLENYGRKGSASQVPSQAPPSRVPEIISPTYRPIGRYTLWETGKGQAPGPSRSSSPGRDGMNSKSAQGLAGLRNLGNTCFMNSILQCLSNTRELRDYCLQRLYMRDLHHGSNAHTALVEEFAKLIQTIWTSSPNDVVSPSEFKTQIQRYAPRFVGYNQQDAQEFLRFLLDGLHNEVNRVTLRPKSNPENLDHLPDDEKGRQMWRKYLEREDSRIGDLFVGQLKSSLTCTDCGYCSTVFDPFWDLSLPIAKRGYPEVTLMDCMRLFTKEDVLDGDEKPTCCRCRGRKRCIKKFSIQRFPKILVLHLKRFSESRIRTSKLTTFVNFPLRDLDLREFASENTNHAVYNLYAVSNHSGTTMGGHYTAYCRSPGTGEWHTFNDSSVTPMSSSQVRTSDAYLLFYELASPPSRM

SEQ ID NO: 3 - protein, 347 aa, Artificial Sequence
  catalytic domain of USP2
NSKSAQGLAGLRNLGNTCFMNSILQCLSNTRELRDYCLQRLYMRDLHHGSNAHTALVEEFAKLIQTIWTSSPNDVVSPSEFKTQIQRYAPRFVGYNQQDAQEFLRFLLDGLHNEVNRVTLRPKSNPENLDHLPDDEKGRQMWRKYLEREDSRIGDLFVGQLKSSLTCTDCGYCSTVFDPFWDLSLPIAKRGYPEVTLMDCMRLFTKEDVLDGDEKPTCCRCRGRKRCIKKFSIQRFPKILVLHLKRFSESRIRTSKLTTFVNFPLRDLDLREFASENTNHAVYNLYAVSNHSGTTMGGHYTAYCRSPGTGEWHTFNDSSVTPMSSSQVRTSDAYLLFYELASPPSRM

SEQ ID NO: 4 - protein, 644 aa, Homo sapiens
MDDIADRMRMDAGEVTLVNHNSVFKTHLLPQTGFPEDQLSLSDQQILSSRQGHLDRSFTCSTRSAAYNPSYYSDNPSSDSFLGSGDLRTFGQSANGQWRNSTPSSSSSLQKSRNSRSLYLETRKTSSGLSNSFAGKSNHHCHVSAYEKSFPIKPVPSPSWSGSCRRSLLSPKKTQRRHVSTAEETVQEEEREIYRQLLQMVTGKQFTIAKPTTHFPLHLSRCLSSSKNTLKDSLFKNGNSCASQIIGSDTSSSGSASILTNQEQLSHSVYSLSSYTPDVAFGSKDSGTLHHPHHHHSVPHQPDNLAASNTQSEGSDSVILLKVKDSQTPTPSSTFFQAELWIKELTSVYDSRARERLRQIEEQKALALQLQNQRLQEREHSVHDSVELHLRVPLEKEIPVTVVQETQKKGHKLTDSEDEFPEITEEMEKEIKNVFRNGNQDEVLSEAFRLTITRKDIQTLNHLNWLNDEIINFYMNMLMERSKEKGLPSVHAFNTFFFTKLKTAGYQAVKRWTKKVDVFSVDILLVPIHLGVHWCLAVVDFRKKNITYYDSMGGINNEACRILLQYLKQESIDKKRKEFDTNGWQLFSKKSQEIPQQMNGSDCGMFACKYADCITKDRPINFTQQHMPYFRKRMVWEILHRKLL

SEQ ID NO: 5 - protein, 230 aa, Artificial Sequence
  catalytic domain of SENP1
DSEDEFPEITEEMEKEIKNVFRNGNQDEVLSEAFRLTITRKDIQTLNHLNWLNDEIINFYMNMLMERSKEKGLPSVHAFNTFFFTKLKTAGYQAVKRWTKKVDVFSVDILLVPIHLGVHWCLAVVDFRKKNITYYDSMGGINNEACRILLQYLKQESIDKKRKEFDTNGWQLFSKKSQEIPQQMNGSDCGMFACKYADCITKDRPINFTQQHMPYFRKRMVWEILHRKLL

(19) United States
(12) Patent Application Publication
(10) Pub. No.: US 2014/0309141 A1
(43) Pub. Date: Oct. 16, 2014

(54) Methods of Screening for Inhibitors of Enzymes

(71) Applicant: Progenra, Inc., Malvern, PA (US)

(72) Inventors: Craig Leach, Ardmore, PA (US); James Strickler, Audubon, PA (US); Michael Eddins, West Chester, PA (US)

(21) Appl. No.: 14/233,537

(22) PCT Filed: Sep. 21, 2012
(86) PCT No.: PCT/US2012/056488

§ 371 Date: Jan. 17, 2014

Related U.S. Application Data
(60) Provisional application No. 61/537,283, filed on Sep. 21, 2011.

(51) Int. Cl. C12Q 1/37; C12Q 1/66; C12N 9/64

(52) U.S. Cl. CPC C12Q 1/37; C12N 9/6472; C12Q 1/66
USPC 506/11; 435/23; 536/23.2; 435/320.1; 435/226; 435/8; 435/7.4; 252/182.12

(57) ABSTRACT

Methods and compositions for detection of the modulators of proteolytic enzymes, particularly cysteine proteases, are disclosed.

This application claims priority under 35 U.S.C. §119(e) to U.S. Provisional Patent Application No. 61/537,283, filed on Sep. 21, 2011. The foregoing application is incorporated by reference herein.

### FIELD OF THE INVENTION

The present invention relates to the field of high throughput screening. More specifically, the instant invention provides compositions and methods for screening for modulators of enzymatic activity, particularly cysteine proteases and ubiquitin related enzymes with a cysteine residue at the catalytic site.

### BACKGROUND OF THE INVENTION

Several publications and patent documents are cited throughout the specification in order to describe the state of the art to which this invention pertains. Full citations of these references can be found throughout the specification. Each of these citations is incorporated herein by reference as though set forth in full.

A major problem in the discovery of small molecule modulators of enzymes, particularly proteases such as cysteine proteases, is the general reactivity of some small molecules with amino acid residues in the active site of proteases. When testing small molecules for activity versus a small molecule library in high throughput screening (HTS) these chemically reactive compounds will be preferentially discovered and prevent the discovery of quality inhibitors. Currently, researchers will use counter-screening methods to identify and discard chemically reactive compounds from further follow-up. This methodology does not diminish the discovery of these compounds in the primary screen and is laborious and time-consuming. Improved and more efficient methods for discovering modulators of enzymes and proteases are desirable.

### SUMMARY OF THE INVENTION

In accordance with the instant invention, methods for screening for modulators of an enzyme, particularly where the enzyme comprises an active site cysteine, are provided. In a particular embodiment, the method comprises contacting at least one active site mutant of the enzyme with at least one compound (e.g., small molecule); and measuring the activity of the mutant enzyme in the presence of the compound, wherein a modulation in the activity of the mutant enzyme in the presence of the compound compared to the activity of the mutant enzyme in the absence of the compound indicates that the compound is a modulator of the wild-type enzyme. The method may further comprise synthesizing the active site mutant. The method may also further comprise contacting the wild-type enzyme with the identified compound and measuring activity of the wild-type enzyme in the presence of the compound (e.g., to confirm the modulatory properties of the compound). The active site cysteine may be replaced by any amino acid, particularly serine. In a particular embodiment, the enzyme is a cysteine protease, deubiquinating enzyme, a ubiquitin-like protein (Ubl)-specific proteases (Ulp), a ubiquitin/UBL activating enzyme, ubiquitin/UBL conjugating enzyme, or ubiquitin/UBL ligase.

In accordance with another aspect of the present invention, nucleic acid molecules encoding an amino acid sequence having at least 80%, 85%, 90%, 95% or more identity with SEQ ID NO: 2, 3, 4, or 5 are provided, wherein the active site cysteine has been mutated (e.g., to a serine). In a particular embodiment, the nucleic acid molecule is in a vector (e.g., plasmid). Polypeptides comprising a sequence having at least 80%, 85%, 90%, 95% or more identity with SEQ ID NO: 2, 3, 4, or 5 are provided, wherein the active site cysteine has been mutated (e.g., to a serine). Compositions (e.g., further comprising at least one carrier) and kits comprising at least one active site mutant of the instant invention are also provided.

### BRIEF DESCRIPTION OF THE DRAWINGS

FIG. 1 provides schematics of the three dimensional structure of the active site of wild-type SENP1 (FIG. 1A) and a mutant SENP1 wherein the active site cysteine has been changed to a serine (FIG. 1B).

FIG. 2 provides a graph of the proteolytic activity of the USP2 catalytic mutant (C276S). Various concentrations of USP2 (C276S) were incubated with Ubiquitin-luciferin and the cleavage of ubiquitin from luciferin was detected using DUB-Glo™.

FIG. 3 provides a graph of the proteolytic activity of SENP1 catalytic mutant. SENP1 (C603S) was incubated with SUMO2-luciferin and the cleavage of ubiquitin from luciferin was detected using DUB-Glo™.

FIG. 4 is a graph of a high throughput screen (HTS) of a small molecule library using the USP2 Cys276Ser catalytic mutant.

FIG. 5 provides a graph of the lack of chemical reactivity in USP2 inhibitors identified by screening with a catalytic mutant.

FIGS. 6A and 6B provide the amino acid sequences of full length USP2 (SEQ ID NO: 2) and the catalytic domain of USP2 (SEQ ID NO: 3), respectively, with the active site cysteine underlined.

FIGS. 7A and 7B provide the amino acid sequences of full length SENP1 (SEQ ID NO: 4) and the catalytic domain of SENP1 (SEQ ID NO: 5), respectively, with the active site cysteine underlined.

FIG. 8 provides a graph of the inhibition of wild-type USP2core by compounds identified in FIG. 4.

### DETAILED DESCRIPTION OF THE INVENTION

The discovery of modulators of proteolytic enzymes, particularly cysteine proteases, is complicated by the presence of alkylating/chemically reactive molecules in screening collections. These molecules will inhibit enzymes with a cysteine active site (e.g., cysteine proteases) but are not the most useful starting points for drug/probe discovery. Indeed, these chemically reactive molecules usually do not display specificity due their generic mechanism of action. The instant invention avoids the discovery of alkylating/chemically reactive molecules and eliminates this major hurdle in drug/probe discovery.

Herein, it is demonstrated that the replacement of the catalytic site cysteine of cysteine proteases results in mutants that possess catalytic activity and that these mutant enzymes can be used for the identification of modulators of protease activity of the wild-type (non-mutated) enzyme. The assay does not identify chemically reactive, generic enzyme inhibitors, which is a major obstacle in drug discovery.

In accordance with the instant invention, methods of screening, detecting, and/or identifying modulators of an enzyme are provided. In a particular embodiment, the method comprises contacting at least one active site mutant of the proteolytic enzyme with at least one compound and measuring the activity of the mutant proteolytic enzyme, wherein a modulation in the activity of the mutant proteolytic enzyme in the presence of the compound compared to the activity of the mutant proteolytic enzyme in the absence of the compound indicates that the compound is a modulator of the proteolytic enzyme. The modulator may be an inhibitor or an enhancer. The method may be a high throughput screening assay.

The compound tested by the methods of the instant invention can be any compound (e.g., an isolated compound), particularly any natural or synthetic chemical compounds (such as small molecule compounds (including combinatorial chemistry libraries of such compounds)), extracts (such as plant-, fungal-, prokaryotic- or animal-based extracts), fermentation broths, organic compounds and molecules, inorganic compound and molecules (e.g., heavy metals, mercury, mercury containing compounds), biological macromolecules (such as saccharides, lipids, peptides, proteins, polypeptides and nucleic acid molecules (e.g., encoding a protein of interest)), inhibitory nucleic acid molecule (e.g., antisense or siRNA), and drugs (e.g., an FDA approved drug). In a particular embodiment, the compound is a small molecule.

In a particular embodiment, the enzyme of the instant invention is a proteolytic enzyme or isopeptidase. The full-length enzyme may be used or a fragment comprising the catalytically active domain. The enzyme may be from any organism. In a particular embodiment, the enzyme is of human origin. In some embodiments, the enzyme of the instant invention is an enzyme which comprises an active site cysteine. In a particular embodiment, the enzyme is a Ubiquitin/Ubl Activating Enzymes, Ubiquitin/Ubl Conjugating enzyme, or a Ubiquitin/ubl ligase. In a particular embodiment, the proteolytic enzyme is a cysteine protease. Cysteine proteases have a catalytic mechanism that involves a cysteine sulfhydryl group. Typically, deprotonation of the cysteine sulfhydryl by an adjacent basic amino acid, usually a histidine residue, is followed by nucleophilic attack of the cysteine on the peptide carbonyl carbon. A thioester linking the new carboxy-terminus to the cysteine thiol is a common intermediate of the reaction. Cysteine proteases include, without limitation, deubiquitinases (DUBs), actinidains, papains, cathepsins, caspases, and calpains. Particular examples of cysteine proteases include, without limitation (examples of GenBank Accession Nos. in parentheses): deubiquitinases, cathepsin L (P07711), cathepsin V (O60911), cathepsin K (P43235), cathepsin S (P25774), cathepsin F (Q9UBX1), cathepsin C (P53634), cathepsin H (P09668), cathepsin O (P43234), cathepsin W (P56202), cathepsin Z (Q9UBR2), cathepsin B (P07858), papain (P00784), cruzain (cruzapain; P25779), caspase 1 (P29466), cell death protein 3 (P42573), capase 3 (P42574), caspase 7 (P55210), caspase 6 (P55212), caspase 2 (P42575), caspase 4 (P49662), caspase 5 (P51878), caspase 8 (Q14790), caspase 9 (P55211), caspase 10 (Q92851), caspase 11 (P70343), caspase 12 (O08736), caspase 13 (O75601), caspase 14 (P31944), caspase Nc (Q9XYF4), paracaspase (Q9UDY8), and poliovirus proteinase 3C (Lawon et al. (1991) Proc. Nati. Acad. Sci., 88:9919-9923).

Examples of Ubiquitin/ubl ligases (e.g., E3) include, without limitation (GeneID in parentheses): HECT (Homologous to the E6-AP Carboxyl Terminus) E3 ligases, NEDD4 (4734), NEDD4L (23327), WWP1 (11059), WWP2 (11060), Itch (83737), SMURF1 (57154), SMURF2 (64750), NEDL1 (23072), NEDL2 (57520), HACE1 (57531), HUWE1 (300697), KIAA0317 (9870), HERC1 (8925), HERC2 (8924), HERC3 (8916), HERC4 (26091), HERC5 (51191), HERC6 (55008), E6-AP (UBE3A) (7337), UBE3B (89910), UBE3C (9690), TRIP12 (9320), HECTD1 (25831), HECTD2 (143279), HECTD3 (79654), EDD/UBR5 (51366), G2E3 (55632), and KIAA0614 (283450).

Examples of Ubiquitin/Ubl Conjugating enzymes (e.g., E2) include, without limitation (Gene ID): UBE2A (7319), UBE2B (7320), UBE2C (11065), UBE2D1 (7321), UBE2D2 (7322), UBE2D3 (7323), UBE2D4 (51619), UBE2E1 (7324), UBE2E2 (7325), UBE2E3 (10477), UBE2F (140739), UBE2G1 (7326), UBE2G2 (7327), UBE2H (7328), UBE2I (7329), UBE2J1 (51465), UBE2J2 (118424), UBE2K (3093), UBE2L3 (7332), UBE2L6 (9246), UBE2M (9040), UBE2N (7334), UBE2NL (389898), UBE2O (63893), UBE2Q1 (55585), UBE2Q2 (92912), UBE2R1 (997), UBE2R2 (54926), UBE2S (27338), UBE2T (29089), UBE2U (148581), UBE2V1 (7335), UBE2V2 (7336), UBE2V3 (55293), UBE2W (55284), UBE2Z (65264), BIRC6 (57448), and AKTIP (64400).

Examples of Ubiquitin/Ubl Activating Enzymes (e.g., E1) include, without limitation (GeneID): UBE1/UBA1 (7317), UBA6/UBE1L2 (55236), UBA2 (10054), UBA3 (9039), UBA5 (79876), UBA7 (7318), ATG7 (10533), NAE1 (8883), and SAE1 (10055).

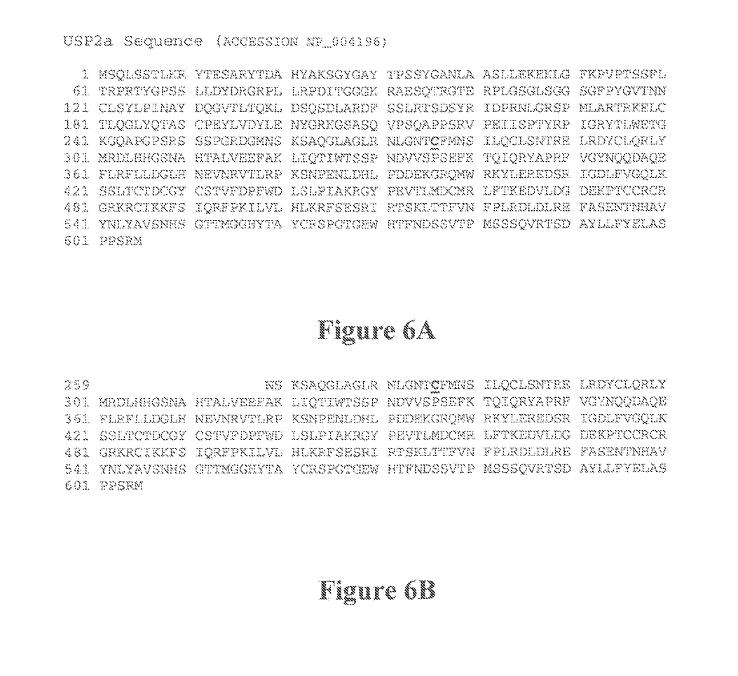

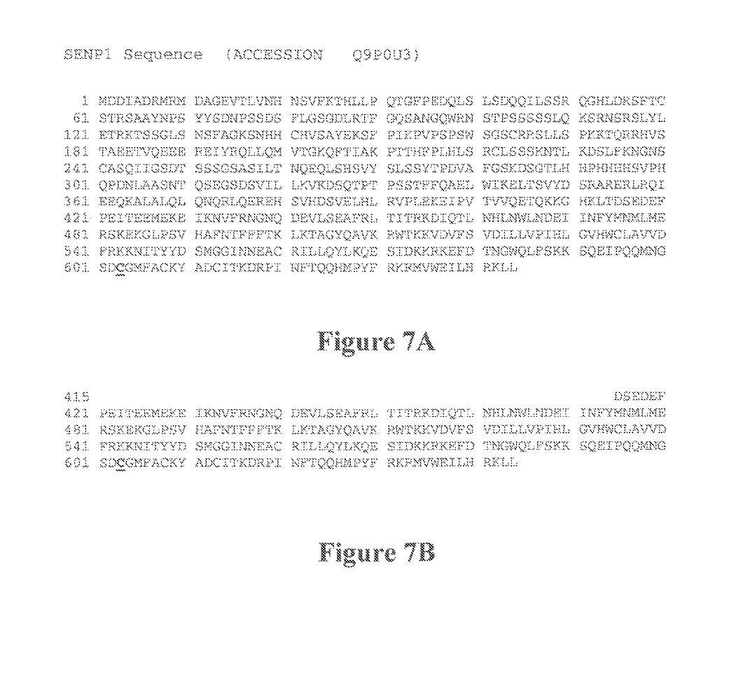

In a particular embodiment, the enzyme of the instant invention is an isopeptidase. Isopeptidases include deubiquitinating enzymes and ubiquitin-like protein (Ubl)-specific proteases (Ulp) (e.g., deSUMOylases). In a particular embodiment, the isopeptidase is a deubiquitinase. Examples of isopeptidases include, without limitation: ULP1, ULP2, SENP1, SENP2, SENP3, SENP5, SENP6 (aka SUSP1, SSP1), SENP7, NEDD8-specific protease 1 (aka DEN1, Nedp1, Prsc2, SENP8), yeast YUH1, mammalian UCH-L1 (aka Park 5), UCH-L3, UCH-L5 (aka UCH37), USP1 (aka UBP), USP2 (aka UBP41), USP2core, USP2a, USP2b, USP3, USP4 (aka UNP, UNPH), USP5 (aka isopeptidase T, ISOT), USP6 (aka TRE2, HRP-1), USP7 (aka HAUSP), USP8 (aka UBPY), USP9, USP9Y (aka DFFRY), USP9X (aka DFFRX), USP10 (aka UBPO, KIAA0190), USP11 (aka UHX1), USP12 (aka USP12L1, UBH1), USP13 (aka ISOT3), USP14 (aka TGT), USP15, USP16 (aka UBP-M), USP18 (aka UBP43, ISG43), USP19 (aka ZMYND9), USP20 (aka VDU1, LSFR3A), USP21, USP22 (aka KIAA1063), USP23, USP24, USP25, USP26, USP27, USP28, USP29, USP30, USP32, USP33 (aka VDU2), USP34, USP35, USP36, USP37, USP38, USP40, USP42, USP44, USP46, USP49, USP51, JosD1 (aka KIAA0063), JosD2 (aka RGD1307305), AMSH, AMSHcore, Ataxin3 (aka ATX3, MJD, MJD1, SCA3, ATXN3), Ataxin3-like, Bap1 (UCHL2 or HUCEP-13), DUB-1, DUB-2, DUB1, DUB2, DUB3, DUB4, CYLD, CYLD1, FAFX, FAFY, OTUB1 (aka OTB1, OTU1, HSPC263), OTUB2 (aka OTB2, OTU2, C14orf137), OUT domain containing 7B (aka OTUD7B, Cezanne), KIAA0797, KIAA1707, KIAA0849, KIAA1850, KIAA1850, KIAA0529, KIAA1891, KIAA0055, KIAA1057, KIAA1097, KIAA1372, KIAA1594, KIAA0891, KIAA1453, KIAA1003, UBP1, UBP2, UBP3, UBP4, UBP5, UBP6, UBP7, UBP8, UBP41, UBP43, VCIP135, Tnfaip3 (aka A20), PSMD14 (aka POH1), COP9 complex homolog subunit 5 (aka CSN5, COPS5, JAB1), and YPEL2 (aka FKSG4, and SARS CoV PLpro). Isopeptidases and their nucleic acid coding sequences are well known to those of skill in the art. For use in certain embodiments, isopeptidases can be isolated or recombinantly produced by methods well known in the art. In particular embodiments, the isopeptidase is USP2 or SENP1. FIGS. 6A and 6B provide the amino acid sequences of full length USP2 and the catalytic domain of USP2, respectively, with the active site cysteine underlined. FIGS. 7A and 7B provide the amino acid sequences of full length SENP1 and the catalytic domain of SENP1, respectively, with the active site cysteine underlined. In a particular embodiment, the isopeptidase comprises SEQ ID NO: 2, 3, 4, or 5.

In a particular embodiment, the proteolytic enzyme/isopeptidase of the instant invention cleaves a ubiquitin or UBL substrate. An exemplary amino acid sequence of ubiquitin is the mature human ubiquitin:

(SEQ ID NO: 1)
MQIFVKTLTG KTITLEVEPS DTIENVKAKI QDKEGIPPDQ
QRLIFAGKQL EDGRTLSDYN IQKESTLHLV LRLRGG,

which is derived by post-translational processing of the naturally occurring human ubiquitin precursor, disclosed at GenBank Accession No CAA44911 (Lund et al., 1985, J. Biol. Chem., 260:7609-7613). In a particular embodiment, the UB or Ubl is the mature form of the protein, i.e., the form of the protein after the precursor has been processed by a hydrolase or peptidase. In particular embodiments, the Ub or Ubl is a mammalian Ub or Ubl, more particularly, a human Ub or Ubl. Ubls include, without limitation, small ubiquitin like-modifier-1 (SUMO), SUMO-2, SUMO-3, SUMO-4, ISG-15, HUB1 (homologous to ubiquitin 1; also known as UBL5 (ubiquitin-like 5)), APG12 (autophagy-defective 12), URM1 (ubiquitin-related modifier 1), NEDD8 (RUB1), FAT10 (also known as ubiquitin D), and APG8. Amino acid sequences of Ubls and nucleic acid sequences encoding Ubls are known in the art. Amino acid and nucleotide sequences of SUMO proteins are provided, for example, in U.S. Pat. No. 7,060,461 and at GenBank Accession Nos. Q12306 (SMT3; amino acids 1-98 is the mature form), P63165 (SUMO1; precursor shown, mature form ends in GG), NM_{—}001005781.1 (SUM01; precursor shown, mature form ends in GG), NP_{—}003343.1 (SUM01; precursor shown, mature form ends in GG), NM_{—}006937.3 (SUMO2; precursor shown, mature form ends in GG), NM_{—}001005849.1 (SUMO2; precursor shown, mature form ends in GG), NM_{—}006936.2 (SUMO3; precursor shown, mature form ends in GG), and NM_{—}001002255.1 (SUMO4; precursor shown, mature form ends in GG). GenBank Accession No. CAI13493 provides an amino acid sequence for URM1. GenBank Accession No. NP_{—}001041706 provides an amino acid sequence for UBL5 (aka HUB1) (amino acids 1-72 represent the mature form). GenBank GeneID No. 4738 and GenBank Accession No. NP_{—}006147 provide amino acid and nucleotide sequences of NEDD8 (RUB1) (precursor shown, mature form ends in LRGG). GenBank Accession No. P38182 provides an amino acid sequence of yeast ATG8 (aka APG8) (precursor shown, mature form ends in FG). GenBank Accession Nos. BAA36493 and P38316 provide amino acid sequences of human and yeast ATG12 (aka APG12), respectively (human precursor shown, mature form ends in FG). GenBank Accession Nos. AAH09507 and P05161 provide amino acid sequences of human and yeast ISG15 ubiquitin-like modifier, respectively (precursors shown, mature form ends in GG). GenBank Accession No. AAD52982 provides an amino acid sequence of ubiquitin D (aka human FAT10, UBD-3, UBD, GABBR1).

In a particular embodiment, the active site mutants comprise a mutation at the catalytic site of the proteolytic enzyme. The mutation may be conservative or non-conservative, particularly conservative. At least one of the amino acids of the active site residues (e.g., catalytic triad) is mutated. In a particular embodiment, the active site mutant comprises a mutation at the active site cysteine. In a particular embodiment, the active site cysteine may be replaced/substituted with a serine, alanine or glycine, particularly a serine.

The activity of the mutated enzyme may be determined by any means appropriate for the enzyme being investigated. For example, when the enzyme is a proteolytic enzyme, the activity of the enzyme may be determined by contacting the proteolytic enzyme with a substrate of the enzyme and detecting the cleavage of the substrate. The cleavage of the substrate may be measured by direct detection of the cleavage products (e.g., detecting the smaller size fragments of the cleaved substrate (e.g., by SDS-PAGE)). In a particular embodiment, the substrate is operably linked to a detectable label to allow for detection of the cleaved substrate. Detectable labels include, for example, chemiluminescent moieties, bioluminescent moieties, fluorescent moieties, radionuclides, isotopes, radisotopes, and metals. In a particular embodiment, the substrate (e.g., Ub or Ubl) is linked at its C-terminus to an enzyme which requires a free amino-terminus for activity such that the enzyme is detectable only upon cleavage of the substrate (see, e.g., U.S. Pat. No. 7,842,460). In a particular embodiment, the substrate comprises at least one fluorescent moiety (e.g., amino-methylcoumarin (AMC) or rhodamine110). Such fluorescent moieties allow for the measurement of increased fluorescence intensity as the fluorophore is liberated from the substrate (e.g., Ub/Ubl molecule; see, e.g., Hassiepen et al., 2007, Analyt. Biochem., 371:201-207 and U.S. Pat. No. 4,336,186). The cleavage of the substrate may also be monitored by modulation (loss) of fluorescence resonance energy transfer (FRET). For example, the substrate may be the Ubiquitin LanthaScreen™ reagent available from Invitrogen (Carlsbad, Calif.; U.S. Patent Application Publication No. 2007/0264678). This assay measures fluorescence resonance energy transfer between a fluorophore at the N-terminus of ubiquitin and a second fluorophore at the C-terminus. In a particular embodiment, luciferase technology may be used to monitor isopeptidase cleavage. In a particular embodiment, the substrate comprises the five C-terminal amino acids of ubiquitin conjugated to an amino-luciferin molecule (DUB-Glo™ (Promega, Inc., Madison, Wis.)). In another embodiment, the substrate comprises the substrate of the enzyme (e.g., Ub or a Ubl) and a luciferase substrate linked to the C-terminus of the Ub or Ubl via an amide linkage (see U.S. patent application Ser. No. 13/157,734). In this assay, the cleavage of the substrate at the C-terminal end of the Ub or Ubl generates free luciferase substrate which can be detecting by luminescence with luciferase, wherein luminescence is indicative of protease activity. The activity of the mutated enzyme can also be measured/detected by measuring the binding of molecules/proteins that bind to the enzyme using biophysical techniques that directly monitor binding of a small molecule/protein to the enzyme such as but not limited to thermal shift assays, isothermal titration calorimetry, surface Plasmon resonance.

Compounds/agents identified as capable of modulating the activity of particular enzyme using the methods of the present invention may useful for the preparation of drugs for the treatment of diseases or conditions associated with a particular enzyme, such as a Ub- or Ubl-specific isopeptidase or its corresponding Ub or Ubl, as well as for further dissecting the mechanisms of action of these enzymes (see, e.g., U.S. patent application Ser. No. 13/168,073).

The present invention also provides kits for screening, detecting, and/or identifying modulators of an enzyme. In some embodiments, the kits comprise one or more active site mutants as described hereinabove (e.g., in a carrier). In some embodiments, the kits may further comprise test compounds (e.g., small molecule library) and/or wild-type enzyme. In some embodiments, the kits may further comprise detectable substrates as explained hereinabove. The kits may optionally comprise instructions. Other optional reagents in the kit can include appropriate buffers for isopeptidase activity. The components of the kits may be contained (individually) in compositions comprising a carrier.

As used herein, “instructions” or “instructional material” includes a publication, a recording, a diagram, or any other medium of expression which can be used to communicate the usefulness of the composition of the invention for performing a method of the invention. The instructions or instructional material of a kit of the invention can, for example, be affixed to a container which contains a kit of the invention to be shipped together with a container which contains the kit. Alternatively, the instructions or instructional material can be shipped separately from the container with the intention that the instructions or instructional material and kit be used cooperatively by the recipient.

### Definitions

As used herein, “proteases,” “proteinases” and “peptidases” are interchangeably used to refer to enzymes that catalyze the hydrolysis of covalent peptidic bonds. Proteases include, without limitation, serine proteases, cysteine proteases, aspartic proteases, threonine and metallo-proteases.

As used herein, the phrase “cysteine protease” refers to a protein or peptide with a protease, peptidase or isopeptidase activity, which is catalyzed in part by a conserved cysteine residue. A catalytic triad may be formed by the cysteine in cooperation with a histidine residue and an aspartic acid residue.

As used herein, a “catalytic triad” or “active site residues” of a cysteine protease refers to a combination of amino acids, typically three amino acids, that are in the active site of a cysteine protease and contribute to the catalytic mechanism of peptide cleavage.

As used herein, the term “catalytic domain” refers to the portion of an enzyme where catalytic action occurs. For cysteine proteases, the catalytic domain comprises the catalytic cysteine.

As used herein, the term “small molecule” refers to a substance or compound that has a relatively low molecular weight (e.g., less than 4,000, particularly less than 2,000). Typically, small molecules are organic, but are not proteins, polypeptides, or nucleic acids, though they may be amino acids or dipeptides.

The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

As used herein, a “conservative” amino acid substitution/mutation refers to substituting a particular amino acid with an amino acid having a side chain of similar nature (i.e., replacing one amino acid with another amino acid belonging to the same group). A “non-conservative” amino acid substitution/mutation refers to replacing a particular amino acid with another amino acid having a side chain of different nature (i.e., replacing one amino acid with another amino acid belonging to a different group). Groups of amino acids having a side chain of similar nature are known in the art and include, without limitation, basic amino acids (e.g., lysine, arginine, histidine); acidic amino acids (e.g., aspartic acid, glutamic acid); neutral amino acids (e.g., glycine, asparagine, glutamine, serine, threonine, tyrosine, cysteine, alanine, valine, leucine, isoleucine, proline, phenylalanine, methionine, tryptophan); amino acids having a polar side chain (e.g., glycine, asparagine, glutamine, serine, threonine, tyrosine, cysteine); amino acids having a non-polar side chain (e.g., alanine, valine, leucine, isoleucine, proline, phenylalanine, methionine, tryptophan); amino acids having an aromatic side chain (e.g., phenylalanine, tryptophan, histidine); amino acids having a side chain containing a hydroxyl group (e.g., serine, threonine, tyrosine), and the like.

As used herein, “modulate” and “capable of modulating”, in reference to a test agent or agent, includes agents that can increase/enhance or inhibit/decrease/diminish the activity of a particular enzyme. Therefore, screening methods of the present invention are useful for identifying agents that can increase/enhance or inhibit/decrease/diminish the activity of a particular enzyme.

A “carrier” refers to, for example, a buffer, diluent, adjuvant, preservative (e.g., benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent, filler, disintegrant, lubricating agent, binder, stabilizer, or vehicle with which an active agent of the present invention can be contained.

The following example is provided to illustrate various embodiments of the present invention. The example is illustrative and is not intended to limit the invention in any way.

### EXAMPLE

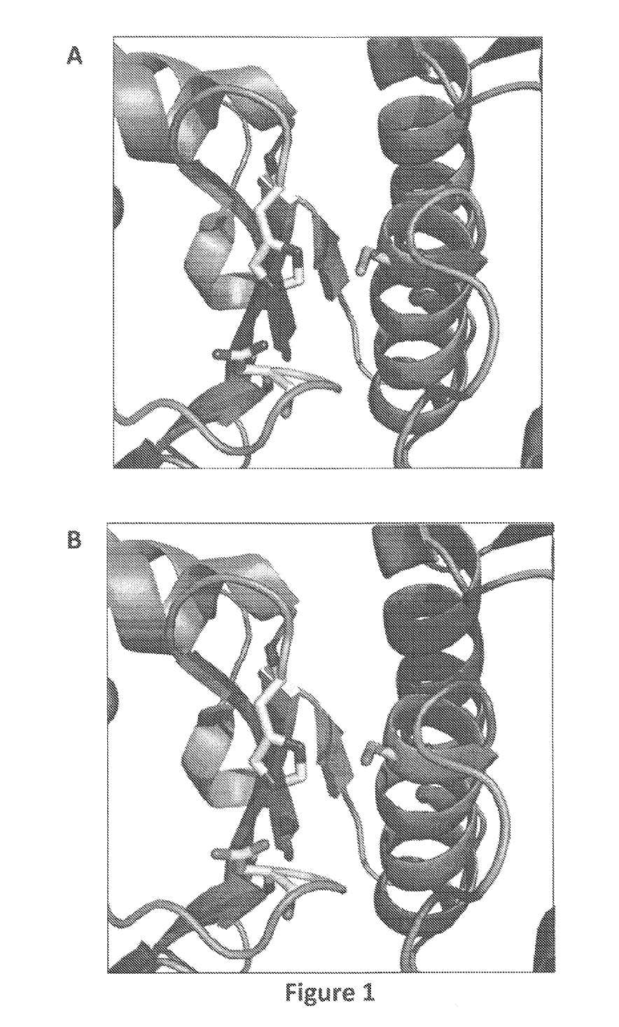

Cysteine to serine mutations in USP2 and SENP1 were prepared. FIG. 1 provides images of the three-dimensional structure of the active site of wild-type SENP1 (FIG. 1A) and the cysteine to serine mutant (FIG. 1B). The activity of these mutated enzymes was determined. Various concentrations of USP2 (C276S) were incubated with 100 nM Ubiquitin-luciferin (LifeSensors, Malvern, Pa.) in 50 mM HEPES, pH 7.5, 10 mM DTT, 0.1% Prionex (Sigma-Aldrich). Activity was measured using the DUB-Glo™ assay system (Promega) according to the manufacturer's instructions with the exception that the DUB-Glo™ was diluted 1:4. The signal in relative luminescence units (RLUs) was measured at 60 minutes from reaction assembly on an Envision multi-label plate reader (Perkin Elmer). SENP1 (C603S) was incubated with SUMO2-luciferin (LifeSensors, Malvern, Pa.) in 50 mM HEPES, pH 7.5, 10 mM DTT, 0.1% Prionex (Sigma-Aldrich). Activity was measured using the DUB-Glo™ assay system (Promega) according to the manufacturer's instructions. The signal in relative luminescence units (RLUs) was measured at 60 minutes from reaction assembly on an Envision multi-label plate reader (Perkin Elmer).

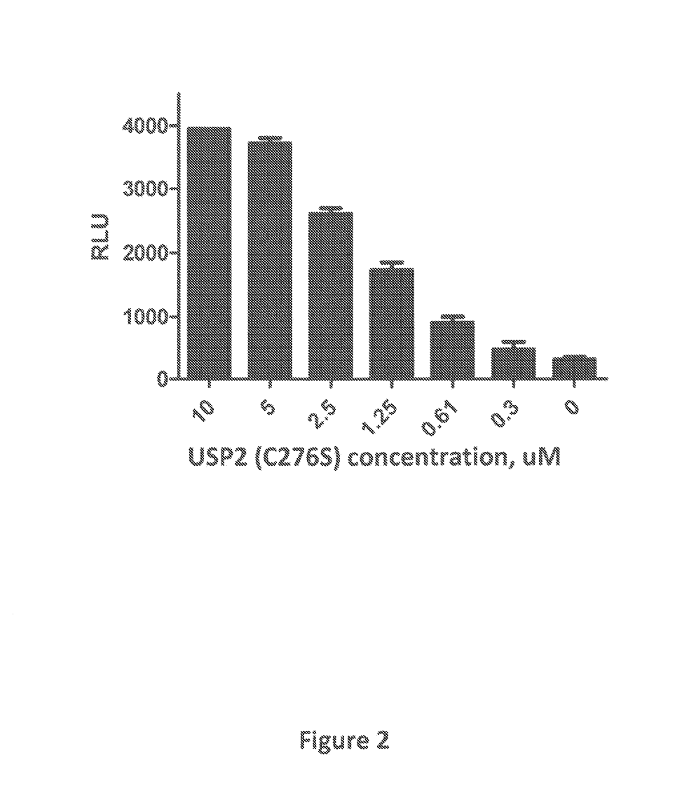

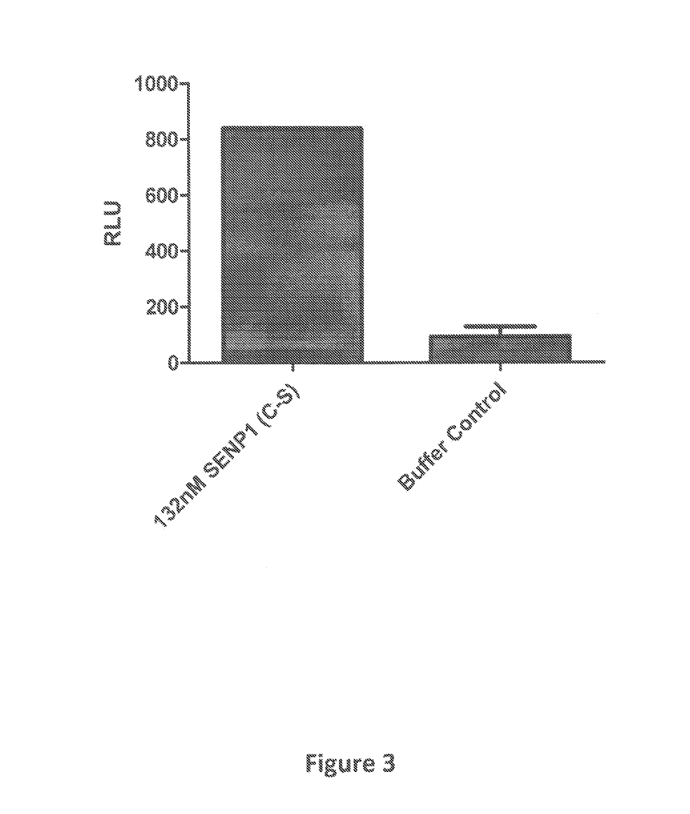

FIGS. 2 and 3 show that the USP2 catalytic mutant (C276S) and the SENP1 catalytic mutant (C603S) retained catalytic activity—particularly, the ability to cleave a ubiquitin-luciferin substrate or SUMO2-luciferein substrate, respectively. This activity allows for using the catalytic mutants in high throughput screening assays.

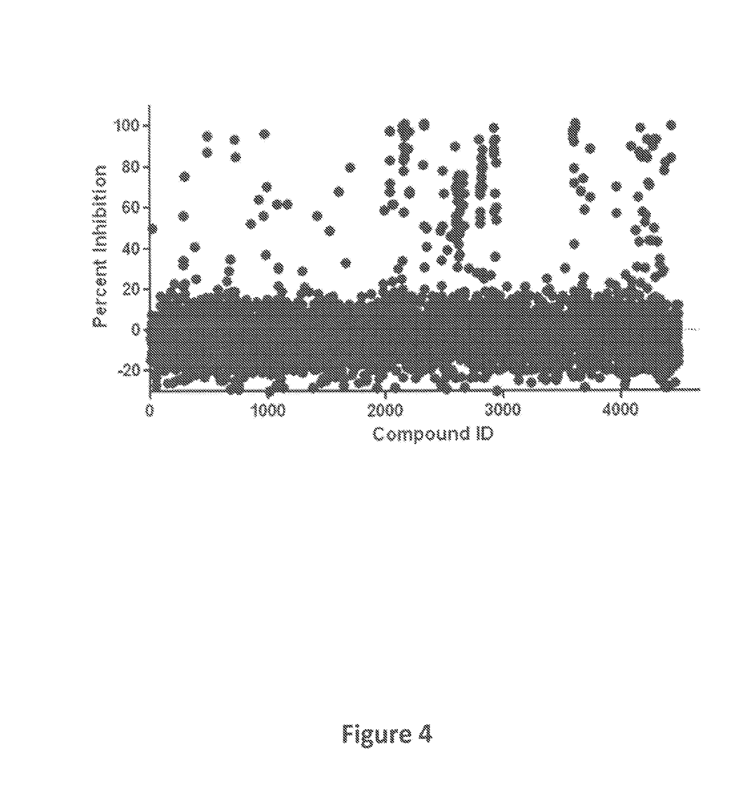

A collection of 5000 small molecules was screened to identify inhibitors of USP2 using the cysteine-serine mutant of USP2 with Ub-luciferin as a substrate. Briefly, 0.5 uL of a stock 5 mM concentration of small molecule was added to the wells of a 384-well white polypropylene plate. 40 uL of buffer containing 12 uM USP2 (C276S) was added to each well except for a small number of wells which served as controls. 10 uL of 1:4 Diluted DUB-GLO™ reagent mixed with 1000 nM Ub-luciferin was added to each well. The signal in relative luminescence units (RLUs) was measured at 60 minutes from reaction assembly on an Envision multi-label plate reader (Perkin Elmer). Percent inhibition was determined for each compound using wells containing vehicle (DMSO) and wells not containing USP2 (C276S) as controls. As seen in FIG. 4, many compounds having up to 100% inhibitory activity were identified.

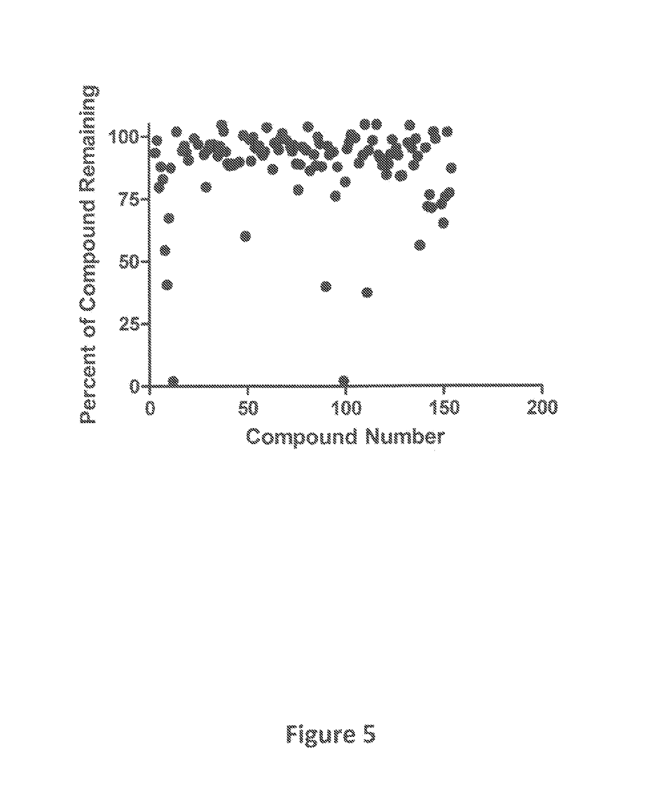

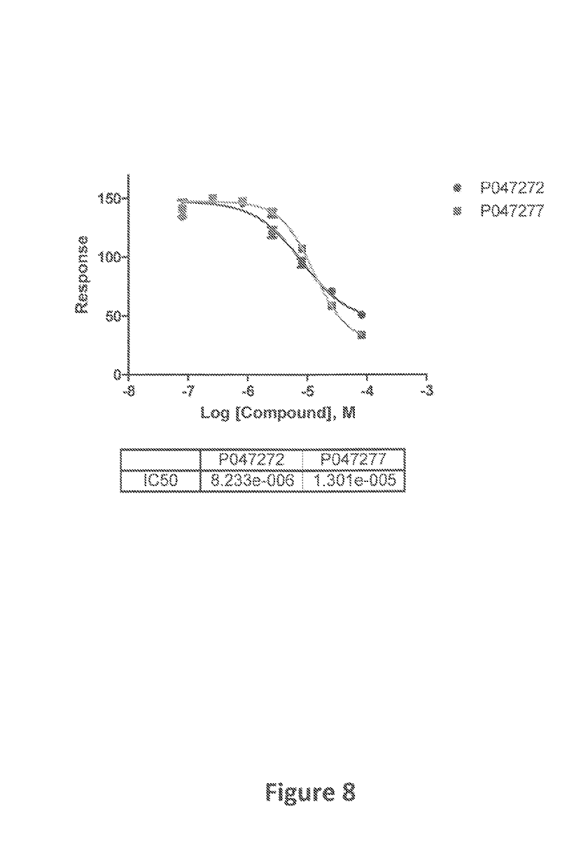

The compounds demonstrating inhibition of mutant USP2 were then tested for chemical reactivity using a standard glutathione assay. Briefly, each small molecule was incubated for 1 hour in 5 mM reduced glutathione (Sigma) in PBS. Samples were analyzed by LC-MS after 0 minutes and 60 minutes incubation at room temperature. The quantity of parent compound was determined at each time point and the loss of compound is interpreted as reactivity with glutathione. This counter-screen demonstrated that the vast majority of compounds identified using this new screening format have minimal chemical reactivity (FIG. 5). The small molecules which inhibit the catalytic mutant of USP2 inhibit were also determined to inhibit the wild-type enzyme (FIG. 8), thus demonstrating the utility of the catalytic mutant strategy for the identification of modulators of the wild-type enzyme. Serial dilutions of small molecules were prepared in DMSO. 0.5 uL of compound was added to the wells of a 384-well polypropylene plate. 40 uL of 12 nM USP2 (WT) was added to each well. DMSO and NEM were utilized as controls. Reactions were started by the addition of 10 uL of 1000 nM IQF Diubiquitin, K4804 (LifeSensors, Malvern, Pa.). Fluorescence was monitored using a BioTek® Synergy Fluorescence Plate Reader.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for screening for modulators of an enzyme wherein said enzyme comprises an active site cysteine, said method comprising measuring the activity of at least one active site mutant of said enzyme in the presence of at least one compound,
wherein a modulation in the activity of the mutant enzyme in the presence of the compound compared to the activity of the mutant enzyme in the absence of the compound indicates that the compound is a modulator of the wild-type enzyme.

2. The method of claim 1, wherein said modulator is an inhibitor.

3. The method of claim 1, wherein said mutant enzyme comprises a serine, alanine, or glycine in place of the active site cysteine.

4. The method of claim 3, wherein said mutant enzyme comprises a serine in place of the active site cysteine.

5. The method of claim 1, wherein said enzyme is a cysteine protease.

6. The method of claim 1, wherein said enzyme is selected from the group consisting of deubiquinating enzyme, ubiquitin-like protein (Ubl)-specific proteases (Ulp), ubiquitin/UBL activating enzyme, ubiquitin/UBL conjugating enzyme, and ubiquitin/UBL ligase.

7. The method of claim 1, wherein said compound is a small molecule.

8. The method of claim 1, wherein said enzyme is the catalytic domain of a full-length enzyme.

9. The method of claim 1 further comprising synthesizing said active site mutant.

10. The method of claim 1 further comprising measuring the activity of said wild-type enzyme in the presence of the identified modulator.

11. An isolated nucleic acid molecule encoding an amino acid sequence having at least 80% homology with SEQ ID NO: 2, 3, 4, or 5, wherein the active site cysteine has been mutated.

12. The isolated nucleic acid molecule of claim 11, wherein said active site cysteine has been changed to a serine.

13. A vector comprising the nucleic acid molecule of claim 11.

14. An isolated polypeptide comprising a sequence having at least 80% homology with SEQ ID NO: 2, 3, 4, or 5, wherein the active site cysteine has been mutated.

15. The isolated polypeptide of claim 14, wherein said active site cysteine has been changed to a serine.

16. A composition comprising at least one polypeptide of claim 14 and at least one carrier.

17. A composition comprising at least one nucleic acid molecule of claim 11 and at least one carrier.

18. A kit comprising at least at least one polypeptide of claim 14 or at least one nucleic acid molecule of claim 11 and, optionally, at least one detectable substrate.
